(19) United States
(12) Patent Application Publication
(10) Pub. No.: US 2024/0242344 A1
(43) Pub. Date: Jul. 18, 2024

(54) AUTOMATED ANALYSIS OF COMPUTERIZED MORPHOLOGICAL FEATURES OF CELL CLUSTERS ASSOCIATED WITH MALIGNANCY ON BILE DUCT BRUSHING IMAGES

(71) Applicants: Case Western Reserve University, Cleveland, OH (US); The United States Government as Represented by The Department of Veteran Affairs, Washington, DC (US)

(72) Inventors: Anant Madabhushi, Shaker Heights, OH (US); Shayan Monabbati, Cleveland, OH (US); Kaustav Bera, Cleveland, OH (US)

(21) Appl. No.: 18/466,032

(22) Filed: Sep. 13, 2023

Related U.S. Application Data
(60) Provisional application No. 63/480,386, filed on Jan. 18, 2023.

(51) Int. Cl. G06T 7/00; A61B 10/02; G06T 7/11; G06T 7/40; G16H 50/20

(52) U.S. Cl. CPC G06T 7/0012; A61B 10/02; G06T 7/11; G06T 7/40; G16H 50/20; A61B 2010/0216; G06T 2207/10056; G06T 2207/20081; G06T 2207/30024

(57) ABSTRACT

The present disclosure, in some embodiments, relates to a method. The method includes accessing one or more digitized pathology images of a cell cluster area comprising epithelial cells obtained from a bile duct of a patient having a bile duct stricture. The cell cluster area is segmented to identify segmented nuclei and non-nuclei regions. A plurality of texture features are extracted from the segmented nuclei and the non-nuclei regions. A plurality of nuclear shape features are extracted from the segmented nuclei. The plurality of nuclear shape features and the plurality of texture features are provided to a machine learning model configured to generate a cytological diagnosis of the epithelial cells within the cell cluster area.

### REFERENCE TO RELATED APPLICATION

This application claims the benefit of U.S. Provisional Application No. 63/480,386, filed on Jan. 18, 2023, the contents of which are hereby incorporated by reference in their entirety.

### BACKGROUND

The pancreas is an organ located in the abdomen. The pancreas has an important role in the body in the generation of hormones that support exocrine and endocrine functioning. The pancreas also produces enzymes that aid in the digestion of food. The enzymes produced by the pancreas are provided to the liver, gallbladder, and stomach by way of small tubes called bile ducts. Pancreaticobiliary disorders can include tumors, obstructions, injuries of the pancreas and bile ducts, and/or pancreatic cancer.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a flow diagram of some embodiments of a method of using a machine learning classifier to generate a cytological diagnosis using texture and/or nuclei shape features associated with epithelial cells from a bile duct of a patient.

FIG. 2 illustrates a block diagram corresponding to some embodiments of a bile duct assessment system configured to generate a cytological diagnosis from texture and/or nuclei shape features extracted from a digitized image of bile duct epithelial cells.

FIG. 3 illustrates a block diagram corresponding to some additional embodiments of a bile duct assessment system configured to generate a cytological diagnosis from a digitized image of bile duct epithelial cells.

FIG. 4A illustrates an exemplary elliptical bounding box that may be used to compute nuclear shape features.

FIG. 4B illustrates a table showing some embodiments of exemplary nuclear shape features that may be used to generate a cytological diagnosis of epithelial cells obtained from a bile duct of a patient.

FIG. 5 illustrates a block diagram corresponding to some additional embodiments of a bile duct assessment system configured to generate a cytological diagnosis from bile duct epithelial cells.

FIG. 6 illustrates a flow diagram of some embodiments of a method of training a machine learning classifier to generate a cytological diagnosis using texture and/or nuclei shape features associated with epithelial cells obtained from a bile duct of a patient.

FIG. 7 illustrates a flow diagram of some embodiments of a method of excluding digitized pathology images from an imaging data training set.

FIG. 8 illustrates some embodiments of benign and malignant whole slide images of biliary tract brush cytology samples.

FIG. 9 illustrates a table showing some exemplary diagnostic results of the disclosed method and/or apparatus.

FIG. 10 illustrates a flow diagram of some additional embodiments of a method of using a machine learning classifier to generate a cytological diagnosis.

FIG. 11 illustrates a block diagram corresponding to some additional embodiments of a disclosed method of using a machine learning classifier to generate a cytological diagnosis.

FIGS. 12A-12B illustrate performance metrics associated with some embodiments of a disclosed method and/or apparatus utilizing a texture classifier.

FIGS. 13A-13B illustrate performance metrics associated with some embodiments of a disclosed method and/or apparatus utilizing a nuclear shape classifier.

FIGS. 14A-14C illustrate performance metrics associated with some embodiments of a disclosed method and/or apparatus utilizing a nuclear shape and texture classifier.

FIGS. 15A-15H illustrate graphical depictions of different diagnostic features used in some embodiments of a disclosed method and/or apparatus of generating a cytological diagnosis using texture and nuclear shape features.

FIG. 16 illustrates a block diagram of some embodiments of an apparatus comprising a machine learning model configured to generate a cytological diagnosis using texture and nuclear shape features associated with epithelial cells obtained from a bile duct of a patient.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Bile duct brushing (BDB) procedures are the preferred method of screening for pancreatic cancer (e.g., adenocarcinoma and cholangiocarcinoma) due to the relatively easy accessibility of bile duct lesions and low complication rates compared with biopsies. A BDB procedure involves inserting a cytology brush attached to a catheter into a bile duct of a patient. As the cytology brush runs along walls of a bile duct stricture, the brush collects cells from an epithelium lining covering the bile duct stricture (e.g., a narrowing in the lumen of the bile duct potentially caused by either primary malignancy or invasion occurring in a pancreas or elsewhere in a biliary tree).

Epithelial cells collected by a BDB procedure can be subsequently analyzed to determine a nature of a bile duct stricture. It is important for health care professionals to be able to accurate differentiate between benign and malignant biliary strictures to be able to provide adequate treatment to a patient. This is because being able to diagnose pancreatic and biliary tract cancer at an early stage enables curative treatment to have a higher likelihood of success. However, despite ever-increasing experience with biliary strictures, being able to diagnose pancreatic and biliary tract cancer at an early stage remains a significant clinical challenge for endoscopist, radiologist, and pathologist alike.

Common etiologies of bile duct strictures include infectious processes, intra-biliary lithiasis, adenocarcinoma, and/or previously installed stents. However, diagnosing epithelial cells collected using a BDB procedure remains a challenge for health care professionals due to low cellularity in cell clusters of BDB samples, which tend to be insufficient in making a diagnosis. Furthermore, bile duct brush specimens may present addition confounding qualifies such as inflammatory and reactive backgrounds due to the local effects of a bile duct stricture and/or atypical reactive changes. As a result, a diagnosis of BDB samples often results in large interobserver variability and low sensitivity. For example, in diagnosing BDB samples having cell clusters that present as atypical (e.g., due to inflammation, infection, and/or the like), cytopathologist are able to achieve an interobserver variability ranging between approximately 6% and approximately 64% with a mean of approximately 42%. The large interobserver variability and low sensitivity cause a large amount of uncertainty in patient management (e.g., including chemotherapy and/or surgery such as Whipple procedures).

The present disclosure relates to a method and/or apparatus configured to generate a cytological diagnosis (e.g., to predict a presence of pancreatic and/or biliary tract adenocarcinoma and/or cholangiocarcinomas) using nuclear shape and texture features extracted from digitized images of epithelial cell clusters collected using a bile duct brushing (BDB) procedure. In some embodiments, the method may be performed by accessing one or more digitized pathology images of a cell cluster area comprising epithelial cells obtained from a bile duct of a patient having a bile duct stricture. The cell cluster area is segmented to identify nuclei. A plurality of texture features are extracted from the cell cluster area and a plurality of nuclear shape features are extracted from the nuclei within the cell cluster area. The plurality of texture features and the plurality of nuclear shape features are provided to a machine learning model configured to automatically generate a cytological diagnosis corresponding to the cell cluster area. By operating a machine learning model on nuclear shape and texture features, the disclosed method may provide for a greatly improved accuracy in diagnosing BDB samples over manual cytopathology. For example, the disclosed method may achieve a sensitivity of approximately 68% for data sets having atypical cell clusters (e.g., 68% of cancerous cell clusters will be positively identified) and a sensitivity of approximately 100% for data sets consisting of atypical cell clusters (e.g., cell clusters having cells that cytopathology cannot identify as malignant or benign).

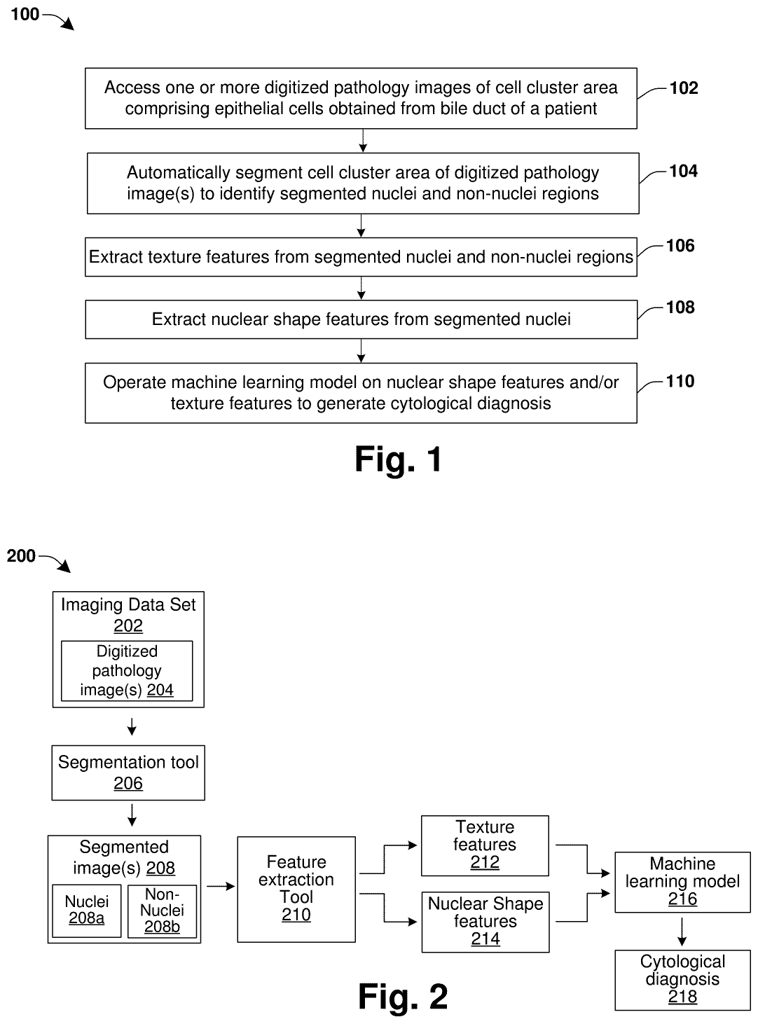

FIG. 1 illustrates a flow diagram of some embodiments of a method 100 of using a machine learning classifier to generate a cytological diagnosis using texture and/or nuclear shape features associated with epithelial cells from a bile duct of a patient

While the disclosed methods (e.g., methods 100, 600, and/or 1000) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 102, one or more digitized pathology images of a cell cluster area are accessed. The cell cluster area comprises epithelial cells (e.g., cells from an epithelium) obtained from a bile duct of a patient having and/or suspected of having a pancreaticobiliary disorder. In some embodiments, the epithelial cells may comprise cells that were collected from a bile duct stricture using a bile duct brushing (BDB) procedure.

At act 104, the cell cluster area of the one or more digitized pathology images is automatically segmented to identify segmented nuclei and non-nuclei regions.

At act 106, a plurality of texture features are extracted from the segmented nuclei and non-nuclei regions.

At act 108, a plurality of nuclear shape features are extracted from the segmented nuclei.

At act 110, the plurality of nuclear shape features and/or the plurality of texture features are provided to a machine learning model configured to generate a cytological diagnosis of the epithelial cells within the cell cluster area.

While the diagnosis of biliary adenocarcinomas is challenging and often results in large interobserver variability and low sensitivity, the disclosed method is able to provide for a relatively high AUC (area under curve) for diagnosis. For example, the disclosed method may achieve an area under curve (AUC) of a receiver operating characteristic (ROC) curve of 0.79. The relatively high accuracy of the disclosed method may provide for better diagnosis of epithelial cells collected by a bile duct brushing (BDB) procedure. Therefore, based upon the cytological diagnosis of epithelial cells within a cell cluster area, a health care professional is able to diagnose pancreaticobiliary disorders (e.g., including pancreatic adenocarcinoma, biliary adenocarcinomas, cholangiocarcinomas, or the like) in a manner that provides for improved treatment of patients suffering from pancreaticobiliary disorders.

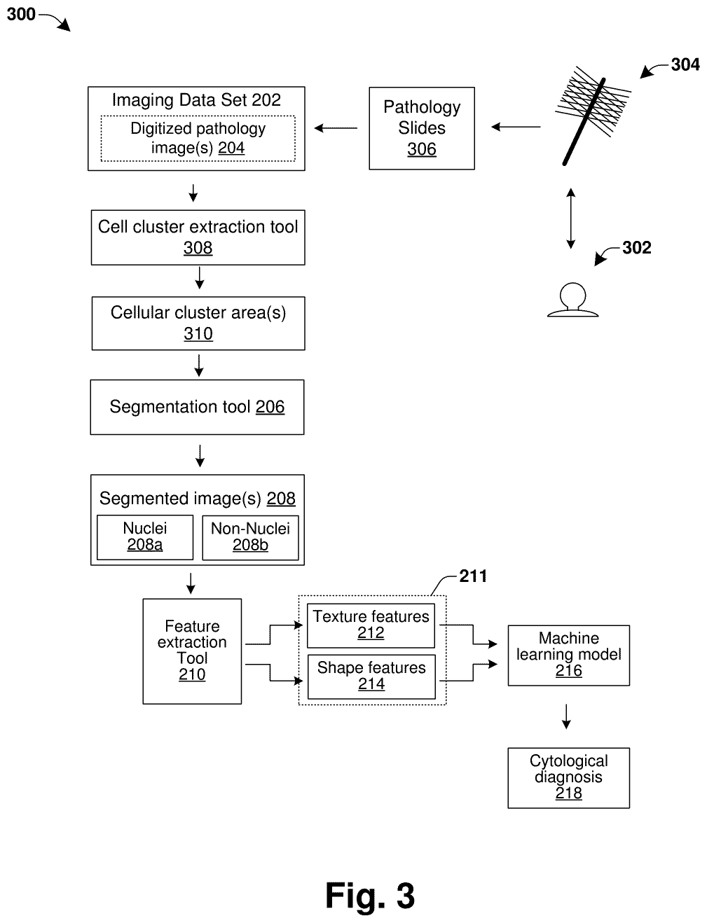

FIG. 2 illustrates a block diagram corresponding to some embodiments of a bile duct assessment system 200 configured to generate a cytological diagnosis from texture and/or nuclear shape features extracted from a digitized pathology image of bile duct epithelial cells.

The bile duct assessment system 200 includes an imaging data set 202 comprising imaging data corresponding to one or more digitized pathology images 204 of one or more patients that have and/or that are suspected of having a pancreaticobiliary disorder (e.g., including biliary adenocarcinomas, cholangiocarcinomas, and/or the like). In some embodiments, the imaging data set 202 may comprise digitized pathology imaging data of epithelial cells collected from a bile duct stricture using a bile duct brushing (BDB) procedure.

A segmentation tool 206 is configured to operate upon the one or more digitized pathology images 204. The segmentation tool 206 is configured to segment the one or more digitized pathology images 204 to identify nuclei and non-nuclei regions within the one or more digitized pathology images 204. In some embodiments, the segmentation tool 206 is configured to segment one or more cell cluster areas in the one or more digitized pathology images 204 to generate one or more segmented images 208 having one or more nuclei 218a and one or more non-nuclei regions 218b (e.g., including cytoplasmic regions, cytoplasm, etc.). In some embodiments, the segmentation tool 206 is configured to perform segmentation on the one or more digitized pathology images 204 using a watershed algorithm to delineate boundary pixels of nuclei (e.g., a Laplacian-of-Gaussian watershed algorithm that identifies boundaries of individual nuclei based on the brightness of edges when an image is transformed into a greyscale space). In some embodiments, the segmentation tool 206 is configured to perform segmentation on all cell cluster areas in the one or more digitized pathology images 204. In some embodiments, the one or more segmented images 208 may comprise or be a binary mask generated using segmented nuclei boundaries outlining a shape of nuclei in each cell cluster area.

A feature extraction tool 210 is configured to extract a plurality of texture features 212 and/or a plurality of nuclear shape features 214 from the one or more segmented images 208. In some embodiments, the plurality of texture features 212 may be extracted from an entire area of the one or more cell cluster areas (e.g., including the one or more nuclei 218a and the one or more non-nuclei regions 218b). Extracting the plurality of texture features 212 from the entire area of the one or more cell cluster areas provides a global quantitative assessment of potentially subvisual diagnostic signals (e.g., including interaction between nuclei and cytoplasm). In some embodiments, the plurality of nuclear shape features 214 may be extracted from the one or more segmented nuclei 208a, but not from the one or more non-nuclei regions 208b.

The plurality of texture features 212 and/or the plurality of nuclear shape features 214 are provided to a machine learning model 216. The machine learning model 216 is configured to operate upon the plurality of texture features 212 and/or the plurality of nuclear shape features 214 to generate a cytological diagnosis 218 corresponding to one or more cell cluster areas within the one or more digitized pathology images 204. In various embodiments, the cytological diagnosis 218 may classify epithelial cells within the one or more digitized pathology images 204 as either benign or malignant.

Traditionally, a cytological diagnosis of cells collected using a BDB procedure achieves a very poor sensitivity (e.g., traditionally only 44% of cancerous cell clusters are positively identified). This means that a rate of false negatives (e.g., a patient having cancer is diagnosed as not having cancer) is problematically high. The disclosed bile duct assessment system 200 is able to provide for a considerably higher sensitivity than that of manual brush cytology. For example, the disclosed bile duct assessment system 200 is able to provide a sensitivity of greater than or equal to approximately 80% (e.g., so that a malignant diagnosis on cytology is correctly diagnosed). The higher sensitivity of the disclosed bile duct assessment system 200 compared with pathologists suggests that it could serve as an initial decision-support tool in low-resource settings. Furthermore, the disclosed bile duct assessment system is able to provide a specificity of greater than 95% or approximately 100% within the atypical category (e.g., so that no false positives are identified). The high specificity is particularly encouraging for such difficult-to-interpret cases since health care professional would rather miss diagnoses a health patient than provide unnecessary treatment (e.g., chemotherapy) to the healthy patient.

The disclosed bile duct assessment system is able to achieve an improved performance over traditional manual assessments because the texture and nuclear shape features that the disclosed bile duct assessment system extracts from the digitized pathology images are at a higher order or higher level than a human can resolve in the human mind or with pencil and paper. For example, epithelial cells from a BDB procedure do not include a texture feature (e.g., Gabor feature) that can be identified by a human.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

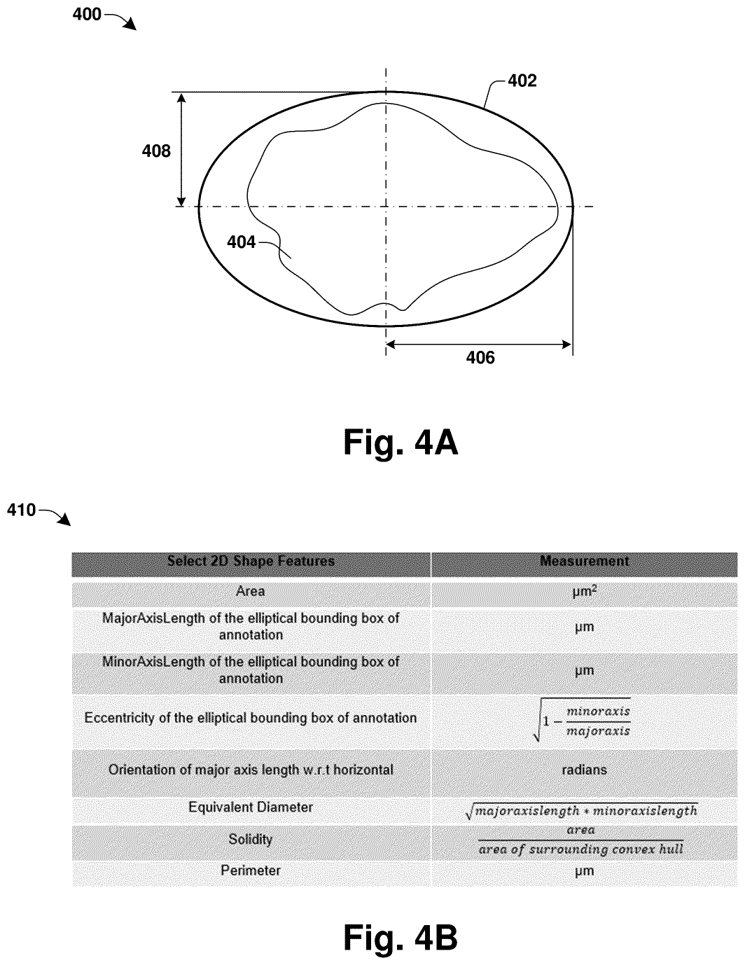

FIG. 3 illustrates a block diagram corresponding to some additional embodiments of a disclosed bile duct assessment system 300 configured to generate a cytological diagnosis from a digitized image of bile duct epithelial cells.

The bile duct assessment system 300 includes an imaging data set 202 comprising imaging data corresponding to one or more digitized pathology images 204 of one or more patients that have and/or that are suspected of having a pancreaticobiliary disorder. In some embodiments, the imaging data set 202 may comprise digitized pathology imaging data of epithelial cells collected from a bile duct stricture using a bile duct brushing (BDB) procedure. The BDB procedure inserts a cytology brush 304 (e.g., a biopsy brush) into a bile duct of a patient 302. When inserted into the bile duct, the cytology brush 304 will collect epithelial cells as it contacts one or more walls of the bile duct. In some embodiments, the cytology brush 304 will collect epithelial cells from a bile duct stricture (e.g., a narrowing in the lumen of the bile duct potentially caused by a primary malignancy, invasion occurring in the pancreas or elsewhere in the biliary tree, or the like). The collected epithelial cells are placed on a transparent slide (e.g., a glass slide) and stained (e.g., using a Papanicolaou stain, an H&E stain, or the like) to generate pathology slides 306 containing brush specimen samples. The pathology slides 306 are subsequently converted to the one or more digitized pathology images 204. In some embodiments, the one or more digitized pathology images 204 may comprise a digitized whole slide image (WSI) of a stained slide (e.g., a WSI of a Papanicolaou stained slide).

A cell cluster extraction tool 308 is configured to automatically identify one or more cell cluster areas 310 within the one or more digitized pathology images 204. The one or more cell cluster areas 310 comprise areas of the one or more digitized pathology images 204 that contain clusters of epithelial cells. In some embodiments, the one or more cell cluster areas 310 may include epithelial cells and surrounding regions (e.g., including cytoplasmic regions, cytoplasm, etc.). The one or more cell cluster areas 310 may be automatically identified using image processing techniques. For example, the one or more cell cluster areas 310 may be automatically identified using a sliding window approach to break a whole slide image into equal sized patches, The patches are then analyzed to detect one or more objects (e.g., 2 objects) per patch with a minimum area filter (e.g., having a size of 100 microns). The remainder of the image is subsequently filtered out.

A segmentation tool 206 is configured to automatically segment the one or more cell cluster areas 310 to identify nuclei and non-nuclei regions within the one or more digitized pathology images 204. In some embodiments, the segmentation tool 206 is configured to segment the one or more digitized pathology images 204 to generate one or more segmented images 208 having one or more segmented nuclei 208a and one or more non-nuclei regions 208b (e.g., including cytoplasmic regions, cytoplasm, etc.). In some embodiments, the one or more segmented images 208 may comprise or be a binary mask generated using segmented nuclei boundaries outlining a shape of nuclei in each cell cluster (e.g., using a watershed algorithm).

A feature extraction tool 210 is configured to extract a plurality of features 211 from the one or more segmented images 208. In some embodiments, the plurality of features 211 may include a plurality of texture features 212 and/or a plurality of nuclear shape features 214. In some embodiments, the plurality of texture features 212 may be extracted from an entire area of the one or more cell cluster areas 310 (e.g., including the one or more segmented nuclei 208a and the one or more non-nuclei regions 208b). In some embodiments, the plurality of nuclear shape features 214 may be extracted from the one or more nuclei 214a, but not from the one or more non-nuclei regions 214b.

In some embodiments, the plurality of texture features 212 may include Gabor features, Law's features, Haralick features, and CoLIAGe (Co-occurrence of Local Anisotropic Gradient Orientations) features. In some embodiments, the plurality of nuclear shape features 214 may comprise two-dimensional shape features of the segmented nuclei 208a. It has been appreciated that the two-dimensional shape features may serve as the basis for differentiating morphologies of benign and malignant cell clusters. In some embodiments, the plurality of nuclear shape features 214 may be determined by forming an elliptical bounding box around each of the nuclei. From the elliptical bounding box, the plurality of nuclear shape features 214 may be measured.

In some embodiments, the plurality of features 211 may comprise a mixture of the plurality of nuclear shape features 214 and the plurality of texture features 212. For example, the plurality of features 211 may comprise a mean CoLIAGe feature (e.g., a mean CoLIAGe information measure 1 that describes textural homogeneity that provides evidence of hyper and hypochromasia and nuclear overcrowding), a mean Haralick contrast entropy, a standard deviation of a CoLIAGe features (e.g., a standard deviation of CoLIAGe information measure 1 that describes a variance in cytoplasmic content and chromatin distribution in malignant nuclei), a mean solidity, and a mean minor axis length. In other embodiments, the plurality of features 211 may comprise a nuclear circularity, a nuclear perimeter, and a nuclear boundary intensity.

The plurality of features 211 are provided to a machine learning model 216, which is configured to operate upon the plurality of features 211 to generate a cytological diagnosis 218 corresponding to cell cluster areas within the one or more digitized pathology images 204. In various embodiments, the cytological diagnosis 218 may classify the cell cluster area and/or the epithelial cells within the one or more digitized pathology images 204 as either benign or malignant. In various embodiments, the machine learning model 216 may comprise a linear discriminant analysis (LDA) classifier, a quadratic discriminant analysis (QDA) classifier, a random forest classifier (e.g., a bagged random forest), or the like.

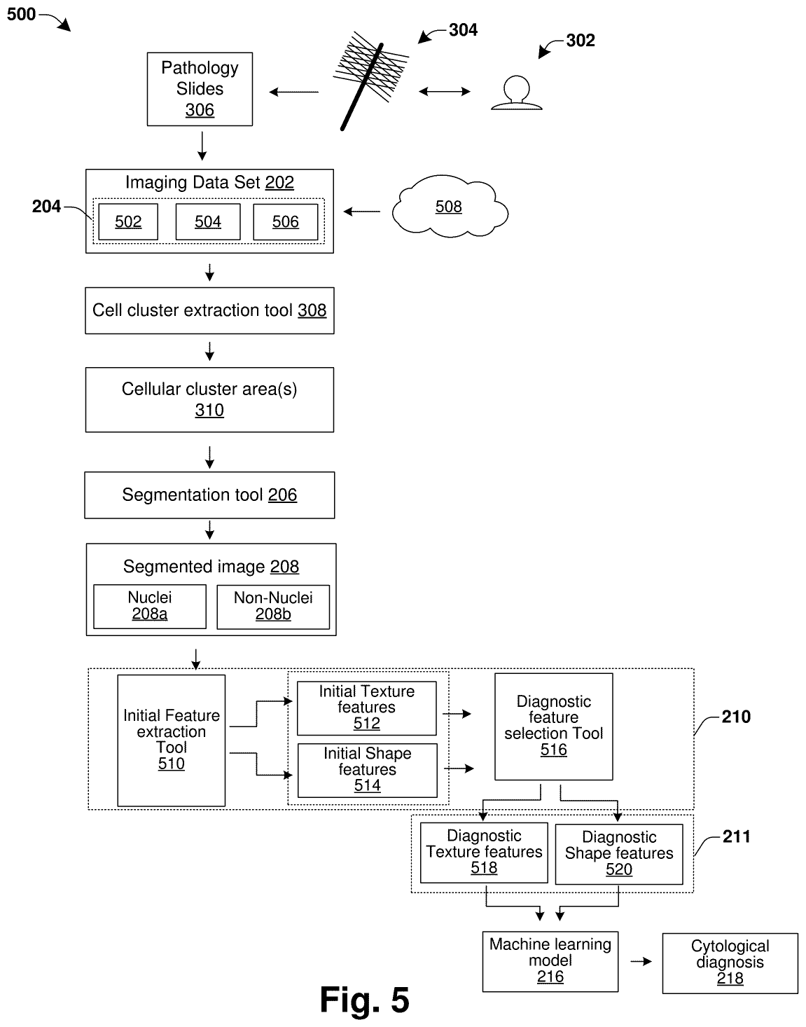

FIG. 4A illustrates an exemplary elliptical bounding box 400 that may be used to compute nuclear shape features (e.g., two-dimensional (2D) nuclear shape features). FIG. 4B shows a table 410 illustrating some exemplary nuclear shape features that may be extracted from segmented nuclei.

As shown in FIG. 4A, the elliptical bounding box 400 comprises an ellipse 402 arranged around a nuclei 404. In some embodiments, the ellipse 402 may be centered upon the nuclei 404 (e.g., upon a centroid of a nuclei 404). Nuclear shape features (e.g., relating to eccentricity) may be determined from the ellipse 402. For example, a major axis length 406 and a minor axis length 408 may be measured using the ellipse 402.

Table 410 of FIG. 4B provides exemplary nuclear shape features. In some embodiments nuclear shape features may comprise one or more of an area of a nuclei (e.g., measured by counting pixels within a nuclei), a major axis length 406 of the elliptical bounding box (e.g., 402 of FIG. 4A), a minor axis length 408 of the elliptical bounding box (e.g., 404 of FIG. 4A), an eccentricity of the elliptical bounding box (e.g., equal to

$$\sqrt{1 - \frac{minor axis length}{major axis length}} ) ,$$

an orientation of a major axis length with respect to a horizontal, an equivalent diameter (e.g., equal
to \sqrt{major axois length*minor axis length}), a solidity (e.g., equal to

$$\frac{area}{area of surrounding convex hull} ) ,$$

and a perimeter of a nuclei (e.g., measured by counting pixels along a perimeter of a nuclei).

In some embodiments, forty nuclear shape features may be extracted from each segmented nuclei. The forty nuclear shape features may be transformed into cluster-level metrics by taking a mean, median, standard deviation, skewness, and kurtosis of nucleus-level features at a cluster level. A nuclei-to-cytoplasm ratio (N:C) may also be computed per cell cluster area by dividing a total nuclear area of the cell cluster area by a non-nuclear area of the cell cluster area. Thus, a total of 41 nuclear shape features may be extracted per cell cluster area.

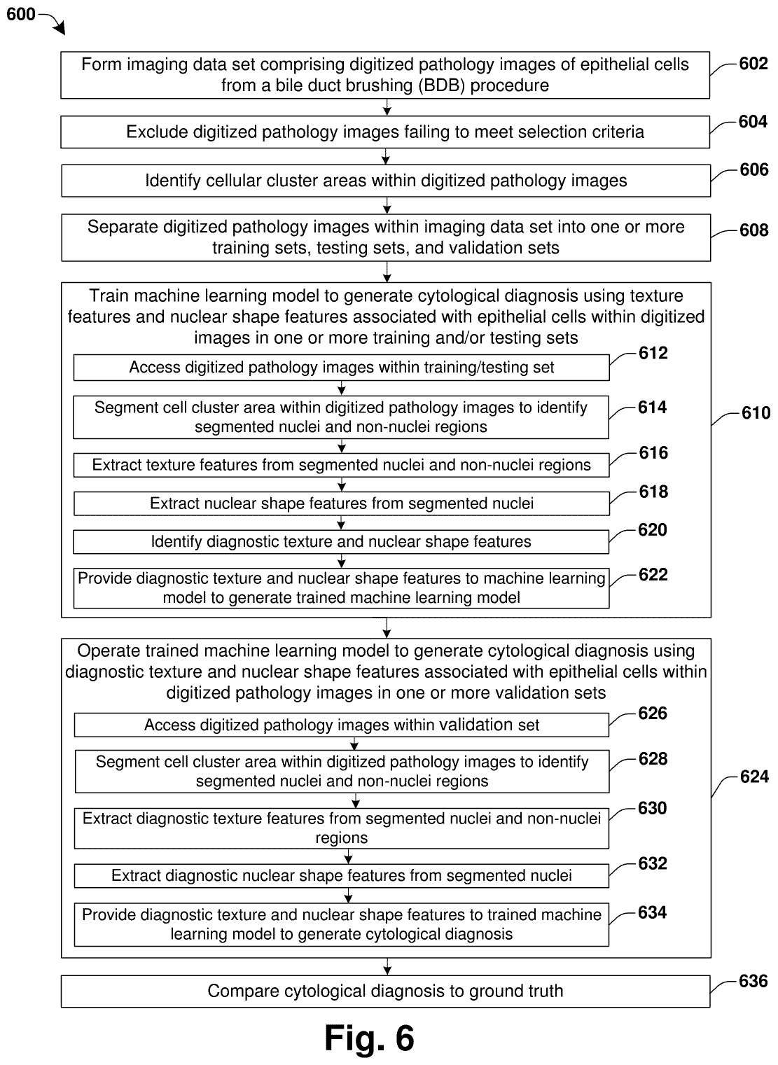

FIG. 5 illustrates a block diagram corresponding to some additional embodiments of a disclosed bile duct assessment system 500 configured to generate a cytological diagnosis from bile duct epithelial cells.

The bile duct assessment system 500 includes an imaging data set 202 comprising imaging data corresponding to one or more digitized pathology images 204 of one or more patients that have and/or that are suspected of having a pancreaticobiliary disorder. In some embodiments, the imaging data set 202 may comprise a training set 502, a testing set 504, and a validation set 506. The training set 502, the testing set 504, and the validation set 506 may be used at different times to train the bile duct assessment system 500. In some embodiments, the imaging data set 202 may be generated by digitizing one or more pathology slides 306 generated from a tissue sample taken from a patient 302. In some additional embodiments, the imaging data set 202 may be generated by accessing images from an online database 508.

A cell cluster extraction tool 308 is configured to identify one or more cell cluster areas 310 within the one or more digitized pathology images 204. In some embodiments, the one or more cell cluster areas 310 may include epithelial cells and surrounding regions (e.g., including cytoplasmic regions, cytoplasm, etc.).

A segmentation tool 206 is operated upon the one or more cell cluster areas 310 to segment the one or more cell cluster areas 310 and identify segmented nuclei and non-nuclei regions within the one or more digitized pathology images 204. In some embodiments, the segmentation tool 206 is configured to segment the one or more digitized pathology images 204 to generate one or more segmented images 208 having one or more segmented nuclei 208a and one or more non-nuclei regions 208b.

A feature extraction tool 210 is configured to extract a plurality of features 211 from the one or more segmented images 208. The plurality of features 211 include one or more texture features and/or one or more nuclear shape features. In some embodiments, an initial feature extraction tool 510 is configured to extract a plurality of initial features 511 from the one or more segmented images 208. The plurality of initial features 511 include initial texture features 512 and/or initial nuclear shape features 514. A diagnostic feature selection tool 516 is then configured to identify a plurality of features 211 from the plurality of initial features 511. The diagnostic feature selection tool 516 is configured to identify the plurality of features 211 as a plurality of diagnostic features that are most diagnostic to separate malignant and benign identification of cell cluster areas. In some embodiments, the plurality of diagnostic features include one or more diagnostic texture features 518 and/or one or more diagnostic nuclear shape features 520. In other embodiments, the diagnostic feature selection tool 516 may utilize a Wilcoxon test, a t test, or mRMR to identify the one or more diagnostic texture features 518 and/or the one or more diagnostic nuclear shape features 520.

It has been appreciated, that at least in-part, the diagnostic features are able to provide for good diagnostic performance due their relation to a cytology of the epithelial cells. For example, it has been appreciated that malignant cell cluster areas tend to exhibit lower textural homogeneity within a nucleus, greater textural entropy around a nuclear membrane, larger variance in cytoplasmic content and chromatin distribution (e.g., identifiable by CoLIAGe features), and longer minor axis lengths, while benign nuclei have more circular and rigid nuclear geometries. The relationship of the diagnostic features to a cytology of the epithelial cells allows for the disclosed bile duct assessment system 500 to enable an accurate cytological diagnosis of epithelial cells obtained by a BDB procedure.

A machine learning model 216 is configured to operate upon the plurality of features 211 to generate a cytological diagnosis 218 corresponding to cell cluster areas within the one or more digitized pathology images 204. In various embodiments, the cytological diagnosis 218 may classify epithelial cells within the one or more digitized pathology images 204 as either benign or malignant. In some embodiments, the machine learning model 216 may be initially trained using the training set 502, adjusted using the testing set 504, and then validated using the validation set 506.

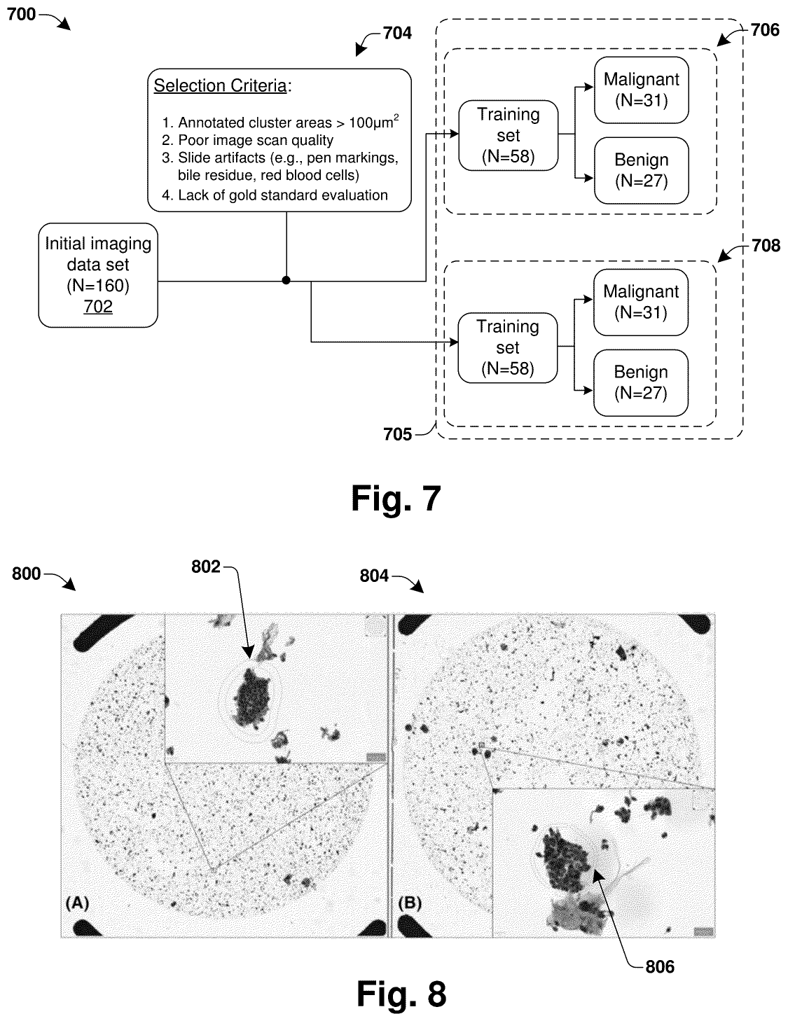

FIG. 6 illustrates a flow diagram of some embodiments of a method 600 of training a machine learning classifier to generate a cytological diagnosis using texture and/or nuclear shape features associated with epithelial cells obtained from a bile duct of a patient.

At act 602, an imaging data set is formed to comprise a plurality of digitized pathology images having cell cluster areas including epithelial cells collected from a bile duct stricture using a bile duct brushing (BDB) procedure.

At act 604, one or more of the digitized pathology images failing to meet selection criteria may be discarded from the imaging data set.

At act 606, cell cluster areas are identified within the plurality of digitized pathology images. In some embodiments, the cell cluster areas may be automatically identified using image process techniques (e.g., a sliding window approach). In other embodiments, an expert cytopathologist may annotate candidate clusters for image-based feature extraction based on the visual quality of cell cluster areas present on each digitized image (e.g., WSI). Based on their domain expertise and discretion, the pathologist selected cell cluster areas may be based on the inclusion of cellularity variety, prominence of nuclear boundaries, and prominence of nucleoli, or the like.

At act 608, the plurality of digitized pathology images within the imaging data set are separated into one or more training sets, one or more testing sets, and/or one or more validation sets.

At act 610, a machine learning model is trained with the one or more training sets and/or the one or more testing sets to generate a cytological diagnosis using texture features and/or nuclear shape features extracted from the one or more digitized pathology images in the one or more training sets and/or the one or more testing sets. In some embodiments, the machine learning model may be trained according to acts 612-622. In some embodiments, the machine learning model may be trained over a plurality of iterations of acts 612-622.

At act 612, digitized pathology images within the one or more training sets and/or the one or more testing sets are accessed.

At act 614, cell cluster areas within the digitized pathology images are segmented to identify segmented nuclei and non-nuclei regions.

At act 616, a plurality of texture features are extracted from the segmented nuclei and the non-nuclei regions.

At act 618, a plurality of nuclear shape features are extracted from the segmented nuclei.

At act 620, diagnostic texture and nuclear shape features are identified.

At act 622, the diagnostic texture and nuclear shape features are provided to a machine learning model configured to generate trained machine learning model. The trained machine learning model is trained to generate a cytological diagnosis of epithelial cells within the cell cluster area.

At act 624, the trained machine learning model is operated to generate a cytological diagnosis using diagnostic texture and/or nuclear shape features extracted from one or more digitized pathology images in the one or more validation sets. In some embodiments, the trained machine learning model operated upon the one or more validation sets according to acts 626-634.

At act 626, one or more digitized validation images within the one or more validation sets are accessed.

At act 628, cell cluster areas within the one or more digitized images are segmented to identify segmented nuclei and non-nuclei regions.

At act 630, the diagnostic texture are extracted from the segmented nuclei and the non-nuclei regions.

At act 632, the diagnostic nuclear shape features are extracted from the segmented nuclei.

At act 634, the diagnostic texture and nuclear shape features are provided to the trained machine learning model to generate a cytological diagnosis of epithelial cells within a cell cluster area.

At act 636, the cytological diagnosis may be compared to a ground truth.

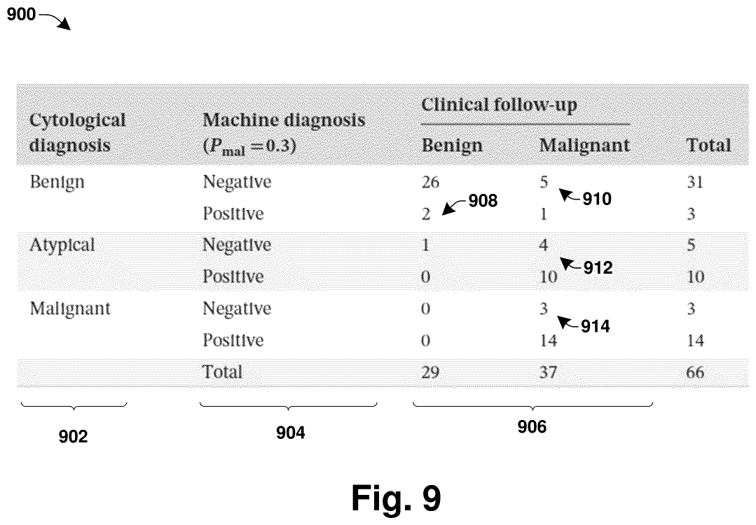

FIG. 7 illustrates a flow diagram of some embodiments of a method 700 of excluding digitized pathology images from an imaging data set.

As shown in method 700, an initial imaging data set 702 is formed to comprise a plurality of digitized pathology images obtained from patients having or suspected of having pancreaticobiliary. The plurality of digitized pathology images respectively comprise one or more cell cluster areas including epithelial cells collected from a bile duct stricture using a BDB procedure.

One or more of the plurality of digitized pathology images that fail to meet a selection criteria 704 are discarded from the initial imaging data set 702 to form an imaging data set 705 (e.g., corresponding to imaging data set 202). In some embodiments, digitized pathology images may be discarded from the initial imaging data set 702 if the digitized pathology images have an annotated cluster area of greater than 100 square microns (μm²), a poor image scan quality, slide artifacts (e.g., pen markings, bile residue, red blood cells, or the like), a lack of corresponding gold standard evaluation, and/or the like.

The plurality of digitized pathology images within the imaging data set 705 that have met the selection criteria are separated into a training set 706 and a validation set 708. In some additional embodiments (not shown), the imaging data set 705 may be further separated into a testing set. In some embodiments, the digitized images within the imaging data set 705 may be separated into the training set 706 and the validation set 708 in a manner that maintains a balance of malignant and benign cluster within each set.

For example, the initial imaging data set 702 may comprise 160 digitized images. Of the 160 digitized images, 36 may be discarded for failing to meet the selection criteria 704, leaving 124 digitized images within the imaging data set 705. The remaining 124 digitized images are divided into the training set 706 comprising 58 digitized images and the validation set 708 comprising 66 digitized images. Within the training set 706, 31 of the digitized images have been identified as having malignant cell clusters and 27 have been identified as having benign cell clusters. Within the validation set 708, 37 of the digitized images have been identified as having malignant cell clusters and 39 have been identified as having benign cell clusters.

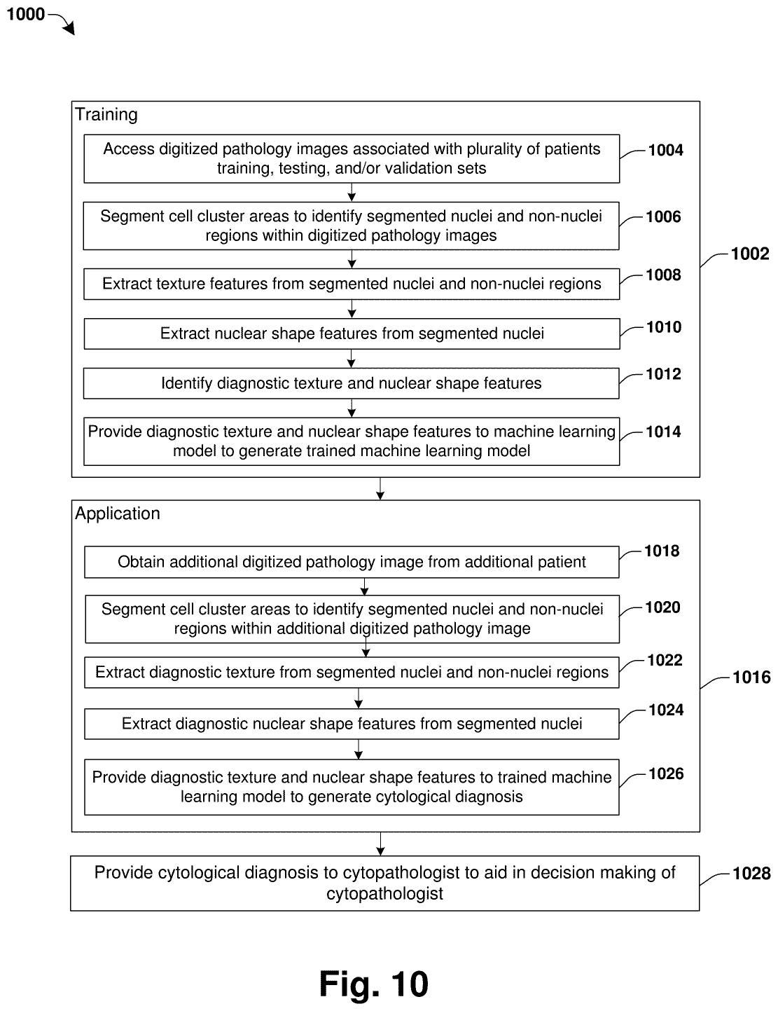

FIG. 8 illustrates some embodiments of a benign whole slide image (WSI) 800 and a malignant WSI 804 of bile duct brush specimens with pathologist annotations.

The benign WSI 800 and the malignant WSI 804 respectively comprise cell cluster areas, 802 and 806. During the disclosed method (e.g., method 600), one or more benign identified clusters may be extracted from the cell cluster area 802 on the benign WSI 800 for image analysis. Similarly, one or more malignantly identified clusters may be extracted from the cell cluster area 806 on the malignant WSI 804 for image analysis. The one or more malignantly identified clusters may be annotated as a ground truth for a supervised approach to classification.

In some embodiments, the annotations may be generated by software (e.g., QuPath) by encircling clusters of interest on the benign WSI 800 and the malignant WSI 804. Digitized cell cluster areas may be subsequently generated by obtaining the bounding box of each annotation, generated by the coordinates of the annotation. The digitized cell cluster areas may be subsequently provided to a disclosed bile duct assessment system (e.g., bile duct assessment system 500), which is configured to operate upon the digitized cell cluster areas to extract features and generate a cytological diagnosis. The cytological diagnosis may be compared to a ground truth to train the disclosed bile duct assessment system.

In some embodiments, the plurality of cell cluster areas within a digitized pathology image may contain both a region of interest (e.g., a cell cluster) and the slide background. In some embodiments, background noise may be removed by filtering pixels with a grayscale intensity greater than a preset threshold from each annotation, determined by the average background intensity of the annotation. In some embodiments, smaller objects, such as stain artifacts, pen markings, bile residue, and individual cells, with an area less than a predefined threshold, may be removed from the annotation as well. In some embodiments, contrast enhancement may be performed on each of the plurality of cell cluster areas by a histogram equalization method to better visually separate the nuclei from the cytoplasm. Performing contrast enhancement also allows for improvement of edge-based segmentation methods.

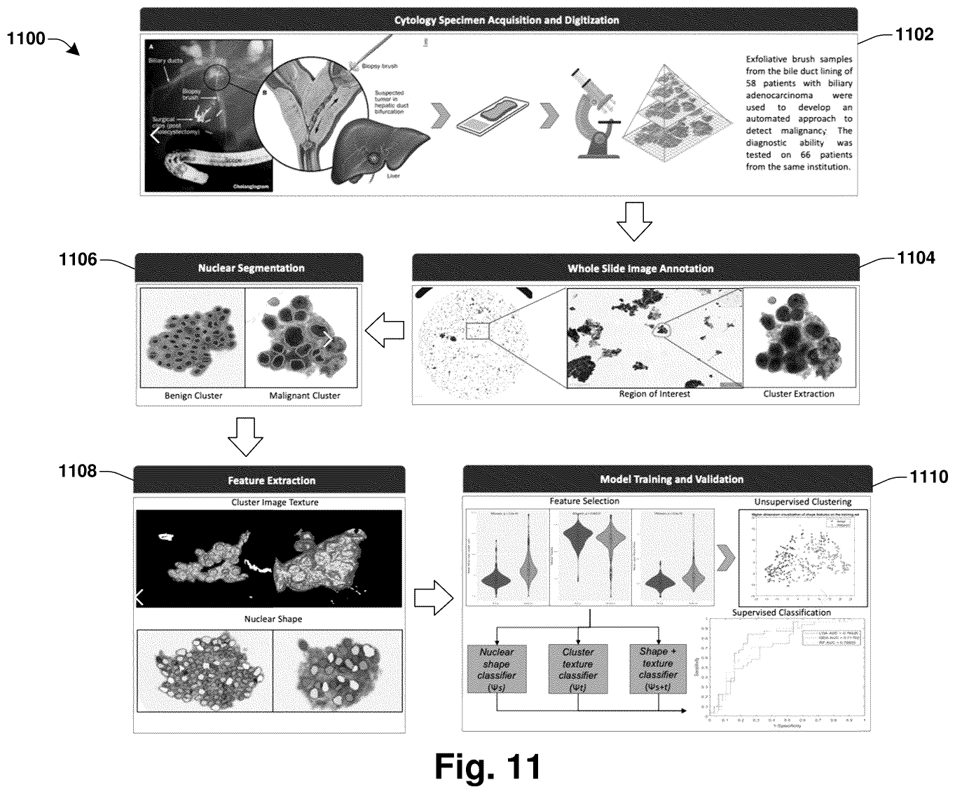

FIG. 9 illustrates a table 900 showing some exemplary diagnostic results of the disclosed method and/or apparatus.

Table 900 illustrates columns corresponding to categories for cytology 902, a machine diagnosis 904 generated by a disclosed bile duct assessment system, and a clinical follow-up 906. The data set of table 900 consisted of 66 patients, of which 37 had malignant BDB specimens and 29 had benign BDB specimens.

When selecting an operating point on a receiver operating curve (ROC) with a threshold on a probability of malignancy of 30%, the machine diagnosis 904 classified two patients as false positives 908 (e.g., two patients having benign clinical follow-up were identified as positive). Twelve patients were classified as false negatives 910-914, two of which were cholangiocarcinoma and could be explained due to bland morphology since both patients tested negative on cytology. Out of the total 15 cases with the atypical diagnosis on cytology, 10 were correctly diagnosed as malignant and one was correctly diagnosed as benign by machine diagnosis. The remaining four cases were benign on machine diagnosis but malignant on clinicopathological follow-up.

The sensitivity of cytology 902 (calculated without atypical cells) was 74%, whereas it was 46% when calculated with atypical cells. However, with the machine diagnosis 904, the sensitivity improved to 68% when atypical cells were included and treated as nonmalignant false negatives. Within the atypical category, the machine-learning model had a specificity of 100% within the atypical category as no false positives were identified. Additionally, 14 out of 17 patients with a malignant diagnosis on cytology were correctly diagnosed. Therefore, in some embodiments a machine learning classifier of the disclosed bile duct assessment system may have a higher specificity in identifying atypical epithelial cells within a cell cluster area than in identifying non-atypical epithelial cells (e.g., benign and/or malignant epithelial cells) within the cell cluster area. This may make the disclosed bile duct assessment system especially useful in identify atypical cell cluster areas (e.g., atypical epithelial cells), which are often been misdiagnosed by cytopathology.

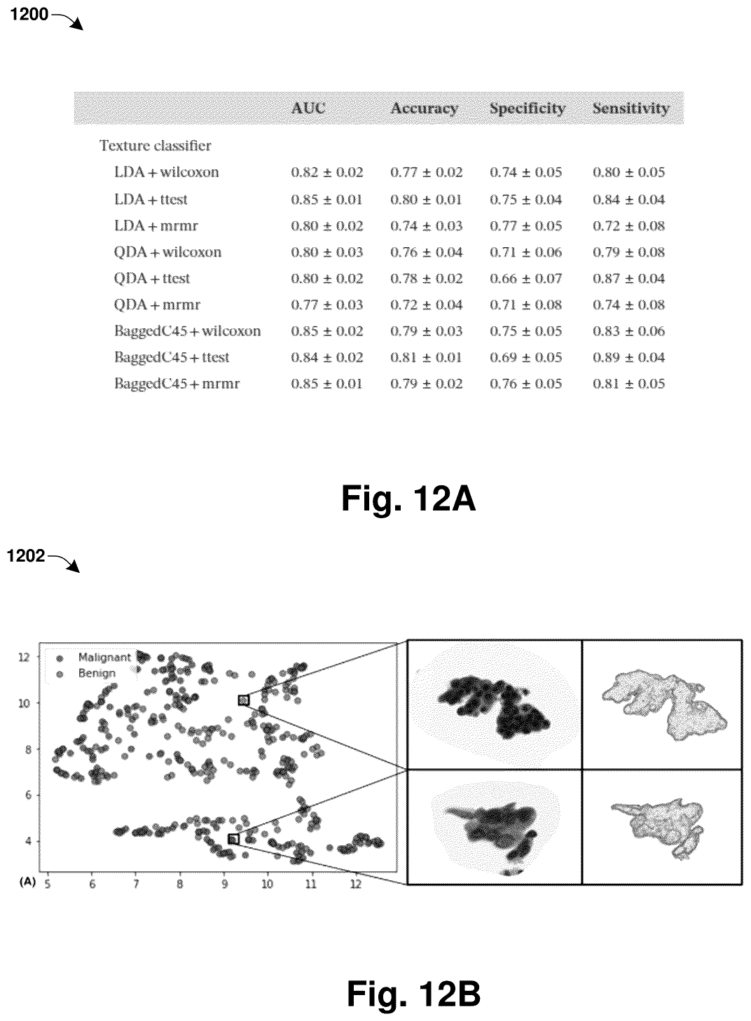

FIG. 10 illustrates a flow diagram of some additional embodiments of a method 100 of using a machine learning model to generate a cytological diagnosis.

The method 1000 comprises a training phase 1002 and an application phase 1016. The training phase 1002 is configured to train a machine learning model to generate a cytological diagnosis. In some embodiments, the training phase 1002 may be performed according to acts 1002-1010.

At act 1004, one or more digitized pathology images from one or more training sets, testing sets, and/or validation sets are accessed.

At act 1006, cell cluster areas within the one or more digitized pathology images are segmented to identify segmented nuclei and non-nuclei regions.

At act 1008, a plurality of texture features are extracted from the segmented nuclei and the non-nuclei regions.

At act 1010, a plurality of nuclear shape features are extracted from the segmented nuclei.

At act 1012, diagnostic texture features and diagnostic nuclear shape features are identified from the plurality of texture features and the plurality of nuclear shape features.

At act 1014, the diagnostic texture features and the diagnostic nuclear shape features are provided to a machine learning model to generate a trained machine learning model. The trained machine learning model is trained to generate a cytological diagnosis of the epithelial cells within cell cluster areas.

The application phase 1016 is configured to utilize the trained machine learning model on an additional digitized pathology image, which was taken from an additional patient using a DBD procedure. In some embodiments, the application phase 1016 may be performed according to acts 1018-1026.

At act 1018, an additional digitized pathology image is obtained from an additional patient. The additional digitized pathology image is obtained by performing a DBD procedure on the additional patient to obtain epithelial cells from the bile duct of the additional patient. The epithelial cells are placed on a transparent slide (e.g., a glass slide) to generate pathology slides. The pathology slides are subsequently converted to one or more digitized pathology images (e.g., one or more digitized whole slide images (WSI)).

At act 1020, cell cluster areas within the additional digitized pathology image are segmented to identify segmented nuclei and non-nuclei regions.

At act 1022, the diagnostic texture features are extracted from the segmented nuclei and the non-nuclei regions.

At act 1024, the diagnostic nuclear shape features are extracted from the segmented nuclei.

At act 1026, the diagnostic texture features and the diagnostic nuclear shape features are provided to the trained machine learning model to generate a cytological diagnosis of the epithelial cells within the cell cluster area.

At act 1028, the cytological diagnosis is provided to a cytopathologist to aid in decision making by the cytopathologist. For example, as shown in FIG. 9, a cytopathologist may be prone to errors in categorizing cells from a DBD procedure (e.g., especially in categorizing atypical cells from a DBD procedure). By providing the cytological diagnosis generated by the disclosed method 1000 to a cytopathologist, the cytopathologist can choose to use the cytological diagnosis to aid in diagnosis (e.g., of atypical cells) to achieve improved results that provide for better overall medical care of a patient.

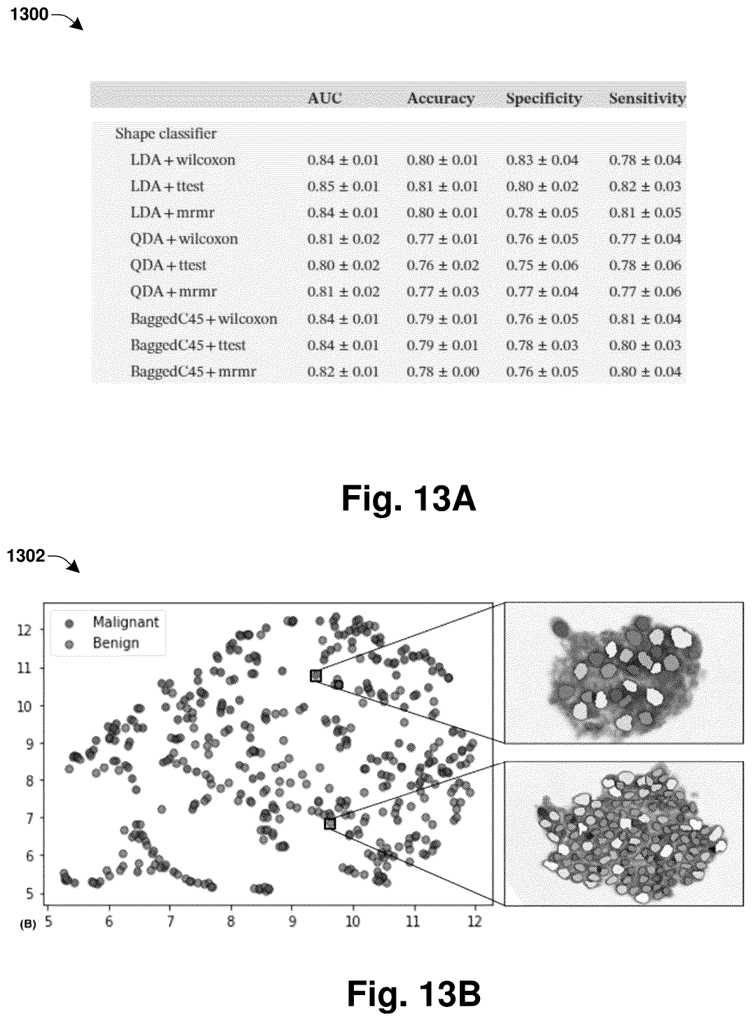

FIG. 11 illustrates a block diagram 1100 corresponding to some embodiments of a method of generating a cytological diagnosis using nuclear shape and texture features associated with epithelial cells obtained from a bile duct of a patient. Although block diagram is described in terms of acts, it will be appreciated that the actions described in block diagram 1100 may also be performed by the disclosed apparatus.

As shown in block diagram 1100, a cytology specimen acquisition and digitization act 1102 is configured to obtain one or more digitized pathology images of epithelial cells collected from a bile duct stricture of a patient using a bile duct brushing (BDB) procedure. In some embodiments, the BDB procedure may be performed by inserting a catheter into a patient's esophagus during an endoscopic procedure (e.g., during an endoscopic retrograde cholangiopancreatography). The catheter is attached to a cytology brush (e.g., a biopsy brush) that is inserted into a bile duct of the patient. When inserted into the bile duct, the cytology brush will collect epithelial cells as it contacts one or more walls of the bile duct. In some embodiments, the cytology brush will collect epithelial cells from a bile duct stricture (e.g., a narrowing in the lumen of the bile duct potentially caused by a primary malignancy, invasion occurring in the pancreas or elsewhere in the biliary tree, or the like). The collected epithelial cells are placed on a transparent slide (e.g., a glass slide) to generate pathology slides (e.g., brush specimen slides). The pathology slides may be magnified (e.g., at 40× magnification) using a microscope and digitized to generate a WSI. In some embodiments, Z-plane settings are not incorporated during digital scanning of each of the pathology slides. The pathology slides are subsequently converted to the one or more digitized pathology images. In some embodiments, the one or more digitized pathology images may comprise a digitized whole slide image (WSI) of a stained pathology slide.

A whole slide image annotation act 1104 is configured to identify a region of interest within the one or more digitized pathology images (e.g., within a WSI). The region of interest extracted from the one or more digitized pathology images contains both a cell cluster area and a slide background. The whole slide image annotation act 1104 is further configured to identify one or more cell cluster areas within the region of interest. In some embodiments, the whole slide image annotation act 1104 may remove background noise from the one or more digitized pathology images by filtering pixels with a grayscale intensity greater than a preset threshold. In some embodiments, the preset threshold may correspond to an average background intensity of the one or more digitized pathology images. In some embodiments, smaller objects, such as stain artifacts, pen markings, bile residue, and individual cells, with an area less than a predefined threshold may be digitally removed from the one or more digitized pathology images. In some embodiments, the whole slide image annotation act 1104 may perform contrast enhancement on the cell cluster areas by a histogram equalization method to better visually separate nuclei from cytoplasm. Performing contrast enhancement also allows for improvement of edge-based segmentation methods.

In some embodiments, the whole slide image annotation act 1104 may deconstruct the one or more digitized pathology images into a plurality of patches (e.g., respectively having a 1000 pixel×1000 pixel area) prior to identification of the region of interest and/or the one or more cell cluster areas. In some embodiments, one or more of the plurality of patches failing to meet one or more selection criteria may be discarded. For example, one or more patches containing over 40% of a background image area may be discarded. The whole slide image annotation act 1104 may identify one or more cell cluster areas in remaining patches by filtering background pixels with grayscale intensity greater than a predefined threshold.

A nuclear segmentation act 1106 may be performed upon the one or more cell cluster areas to generate one or more segmented images that identify segmented nuclei and non-nuclei regions within the cell cluster regions. A segmented image (e.g., a binary mask) may be generated for each cell cluster area by using the segmented nuclei boundaries outlining the nuclei. In some embodiments, the nuclear segmentation act 1106 is configured to perform segmentation using a watershed algorithm that splits clustered objects by first identifying regional minima in a negative distance map of a binary array and then determining boundaries by outwardly growing from regional minima until separating boundaries are reached.

A feature extraction act 1108 may operate upon the one or more segmented images to extract a plurality of features including texture features and nuclear shape features. In some embodiments, before any texture features are extracted, local effects of illumination may be removed by moving a fixed size window (e.g., a 15 pixel×15 pixel window) on each cell cluster area iteratively for each pixel and subtracting the local average for each window from the original image.

In some embodiments, the feature extraction act 1108 may extract different classes of texture features from the one or more segmented images. In some embodiments, the different classes of texture features may include Gabor features, Law's features, Haralick features, and CoLIAGe (Co-occurrence of Local Anisotropic Gradient Orientations) features.

In some embodiments, the Gabor features may be computed from a convolution of a sinusoidal-modulated Gaussian kernel function. These filters may be used to extract dominant-oriented textures within the one or more cell cluster areas. In some embodiments, a total of 48 Gabor features may be extracted across different frequencies and orientations for each cell cluster area. In some embodiments, parameters of the Gabor texture features may include spatial frequencies of {0, 2, 4, 8, 16, or 32} and orientations of θ=0, π/8, π/4, 3π/8, π/2, 5π/8, 3π/4, 7π/8. In some embodiments, the Law's texture features may be computed by convolving a set of 1D line kernels with each cell cluster area. These features aim to identify the level (L), edge (E), spot (S), wave (W), and ripple (R) patterns. In some embodiments, a set of 25 unique 5×5 kernel response maps may be computed by taking the outer product for each possible pair of vectors. In some embodiments, parameters of the Law's texture features may include Level (L5): [1 4 6 4 1], Edge (E5): [−1 −2 0 2 1], Spot (S5): [−1 2 0 −2 1], Wave (W5): [−1 2 0 −2 1], and Ripple (R5): [1 −4 6 −4 1]. In some embodiments, the Haralick features may be computed using a Gray-Level Co-occurrence Matrix (GLCM). The GLCM measures a joint probability of occurrence of any given pair of intensity values in a local window for each pixel. Second-order statistical measures extracted from a GLCM matrix, called Haralick features, describe metrics of pixel intensity contrast, entropy, energy, homogeneity, and correlation. In some embodiments, the Haralick features may contain 13 unique 5×5 kernels, quantized to 64-level intensity. In some embodiments, the CoLIAGe features may be computed using similar kernels as the Haralick features but are combined with the Histogram of Oriented Gradients (HOG) feature descriptors. Gradient magnitudes may be computed for each pixel, and then local dominant orientations are computed by singular value decomposition. The most significant orientations are then determined by computing second-order statistics similar to the Haralick features. In some embodiments, the CoLIAGe features may contain 13 unique 5×5 CoLIAGe kernels convolved with each input image.

In some embodiments, the plurality of texture features may include pixel level texture features and global-level texture features. The pixel level texture features are respectively computed at a pixel level. The global-level texture features may be obtained by taking statistical measures of the pixel level texture features for each cell cluster area. In some embodiments, five statistical measures may be computed for each texture feature for each cell cluster area: mean, median, standard deviation, skewness, and kurtosis. In some embodiments, a total of 495 texture features may be extracted from 99 kernel responses across the entire cell cluster areas. The plurality of texture features may be evaluated at an aggregate level (e.g., over an entire cell cluster area rather than on individual nuclei) to investigate the potential interplay between nuclei and cytoplasm in malignancy.

In some embodiments, the nuclear shape features may comprise two-dimensional nuclear shape features of the segmented nuclei. The two-dimensional nuclear shape features may serve as the basis for differentiating morphologies of benign and malignant cell cluster areas. In some embodiments, the nuclear shape features may be determined by forming an elliptical bounding box around each of the nuclei. From the elliptical bounding box, one or more nuclear shape features may be measured. In some embodiments, forty nuclear shape features may be extracted from each segmented nucleus. A nuclei-to-cytoplasm ratio (N:C) may also be computed per cell cluster area by dividing a total nuclear area of the cell cluster area by a non-nuclear area of the cell cluster area. Thus, a total of 41 nuclear shape features may be extracted per cell cluster area.

A model training and validation act 1110 is configured to receive the nuclear shape features and the texture features. The nuclear shape features and the texture features (e.g., all 41 nuclear shape features and 495 texture features extracted from each cell cluster area) may be ranked on class discriminability to identify diagnostic texture and nuclear shape features. In various embodiments, the nuclear shape features and the texture features may be ranked using a Wilcoxon rank-sum (Mann-Whitney) test, a two-sided t test, a minimum redundancy maximum relevance (mRMR) optimization algorithm, or the like. The Wilcoxon test evaluates the difference in medians rather than means between two distributions. It does not assume normally distributed benign and malignant classes or any prior known distributions. The mRMR scheme identifies and prunes statistically dependent textural features while selecting features that would provide optimized classification performance given these constraints. In some embodiments, a same tool may perform feature extraction and classification.

The diagnostic nuclear shape features and texture features are provided to a machine learning model (e.g., a machine learning classifier). The machine learning model is configured to operate upon the diagnostic nuclear shape features and texture features to classify cell cluster areas from the digitized pathology image as malignant or benign. In some embodiments, the machine learning model may comprise a nuclear shape classifier configured to classify the cell cluster areas based upon the diagnostic nuclear shape features (without texture features). In other embodiments, the machine learning model may comprise a texture classifier configured to classify the cell cluster areas based upon diagnostic texture features (without nuclear shape features). In yet other embodiments, the machine learning model may comprise or a nuclear shape and texture classifier configured to classify the cell cluster areas based upon both diagnostic texture features and nuclear shape features.

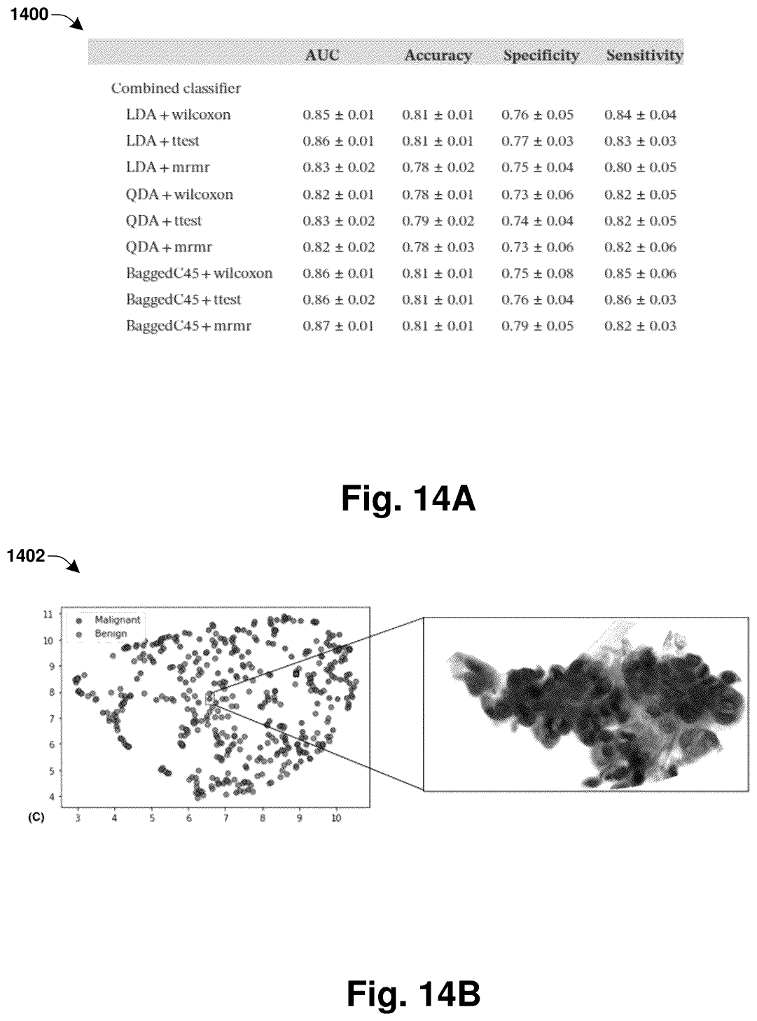

FIGS. 12A-14C illustrate performance metrics associated with different machine learning models (e.g., a nuclear shape classifier, a texture classifier, and a nuclear shape and texture classifier). The performance metrics are shown for a five most diagnostic features selected from each of Wilcoxon, t test, and mRMR trained on LDA, QDA, and random forest classifiers and evaluated on AUC, sensitivity, and specificity.

FIGS. 12A-12B illustrate performance metrics associated with some embodiments of a disclosed method and/or apparatus of generating a cytological diagnosis using a top five diagnostic texture features (e.g., aggregate cluster texture features) for a texture classifier.

The top five diagnostic texture features (e.g., the five most diagnostic texture features) were selected using three different feature selection schemes (e.g., Wilcoxon test, t test, and mRMR) and each set was trained on each of three machine-learning classifiers (e.g., LDA, QDA, and Bagged random forest). The five most diagnostic nuclear shape features include the mean Haralick information measure 2, standard deviation of CoLIAGe correlation, mean CoLIAGe correlation, mean intensity entropy, and median of contrast entropy. The benign clusters exhibit on average a smaller local textural homogeneity than malignant nuclear counterparts. The mean information measure 2 correlation is higher in benign clusters, however, in malignant clusters, a greater variance and mean in the CoLIAGe correlation may be present.

As shown in table 1200 of FIG. 12A, the best performance of a texture classifier in differentiating malignant and benign clusters was provided by the LDA classifier with t test selected features, achieving an AUC of 0.85±0.01, a specificity of 0.75±0.01, and a sensitivity of 0.84±0.04 on 100 iterations of three-fold cross-validation.

FIG. 12B illustrates a UMAP plot 1202 that depicts a diagnostic ability of the top five diagnostic texture features for the texture classifier. The UMAP transformation is a stochastic, nonlinear map that visually depicts a diagnostic ability for the diagnostic texture features. The UMAP plot 1202 represents a cell cluster area of benign samples that form an inner core of a manifold, while the malignant samples form the periphery of the malignant cell cluster areas also exhibit on average greater intensity entropy and lower contrast entropy. The formation of distinct cell cluster areas suggests that the malignant and benign cell cluster areas are separable by selecting a few prominent texture features.

UMAP plot 1202 indicates discrepancies in aggregate texture between benign and malignant cell cluster areas using the top five diagnostic texture features selected from the t test. The top diagnostic texture feature (CoLIAGe information measure 1) is visually depicted as a heat map on the cell cluster areas. Red regions represent a high magnitude of contrast entropy around the nuclei, more so around the perimeter of malignant nuclei, whereas blue regions indicate regions of lower contrast entropy toward the center of benign nuclei.

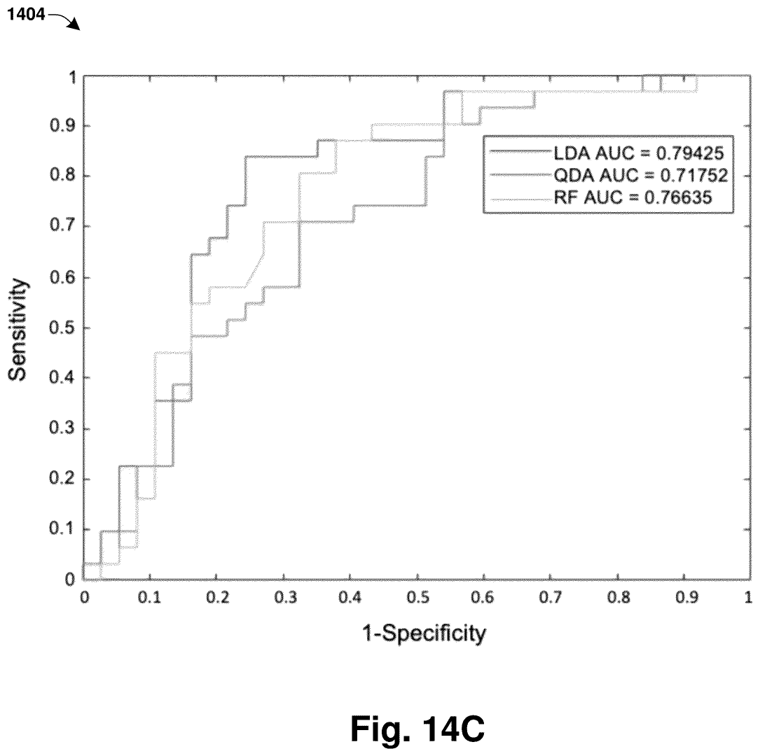

FIGS. 13A-13B illustrate performance metrics associated with some embodiments of a disclosed method and/or apparatus of generating a cytological diagnosis using a top five diagnostic nuclear shape features for a nuclear shape classifier.

The top five diagnostic nuclear shape features were selected using three different feature selection schemes (e.g., Wilcoxon test, t test, and mRMR) and each set was trained on each of three machine-learning classifiers (e.g., LDA, QDA, and Bagged random forest). The top five diagnostic nuclear shape features include a mean nuclear perimeter, a mean solidity, a median minor axis length, a median solidity, and a standard deviation of the nuclear perimeter.

As shown in table 1300 of FIG. 13A, the best performance of a nuclear shape classifier in differentiating malignant and benign clusters was provided by the LDA classifier with features selected from the t test, achieving an AUC of 0.85±0.01, a specificity of 0.80±0.02, and a sensitivity of 0.82±0.03 on 100 iterations of three-fold cross-validation. In comparison to the texture shape, the nuclear shape classifier performed similarly in terms of AUC, however, presented with 5% greater specificity and 2% lower sensitivity.

FIG. 13B illustrates a UMAP plot 1302 that depicts a diagnostic ability of the top five diagnostic nuclear shape features for the nuclear shape classifier. The UMAP plot 1302 shows a separation ability between malignant and benign clusters via the top five diagnostic nuclear shape features. For reference, a visualization of the magnitude of the minor axis length for a benign cell cluster area was compared against a malignant cell cluster area. In general, benign cell cluster areas tend to have on average a smaller minor axis length and a greater solidity (more firm and rigid shape) compared with malignant cell cluster areas. The overlaid color polygons on the nuclei represent the magnitude of difference between benign and malignant clusters for the minor axis length feature. For example, a cool colored polygons (e.g., blue polygons) indicate a relatively large minor axis length that provides for a rounder nucleus in benign nuclei, while warm colored polygons (e.g., red polygons) indicate a relatively small minor axis length that provides for an oblong and/or elliptical nuclei in malignant nuclei.

FIG. 13B also illustrates contrast entropy feature maps corresponding to benign and malignant cluster presentations. This feature includes measures of entropy in the computed gray-level probabilities. The maps reveal that the nuclear boundaries are present with stronger edges compared with the benign cell cluster areas.

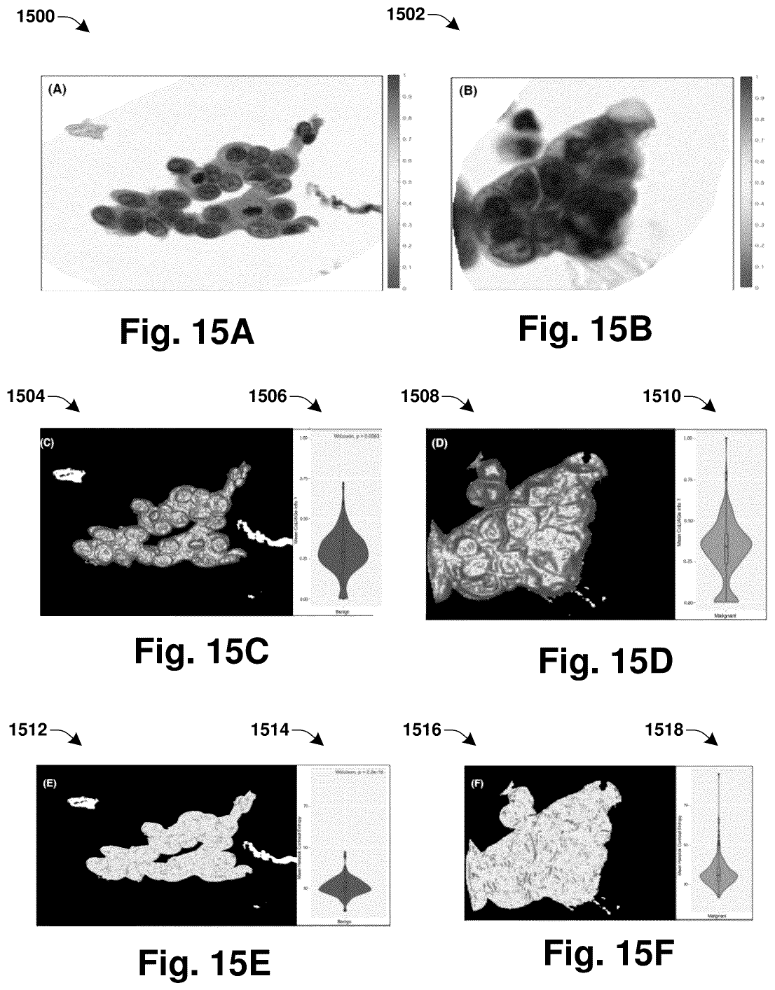

FIGS. 14A-14C illustrate performance metrics associated with some embodiments of a disclosed method and/or apparatus of generating a cytological diagnosis using a diagnostic combination including a top five diagnostic nuclear shape and cluster texture features for a nuclear shape and texture classifier.

The top five diagnostic nuclear shape and cluster texture features were selected using three different feature selection schemes (e.g., Wilcoxon test, t test, and mRMR) and each set was trained on each of three machine-learning classifiers (e.g., LDA, QDA, and Bagged random forest). The top five diagnostic nuclear shape and cluster texture features include a mean contrast entropy, a standard deviation of CoLIAGe correlation information measure 1, a mean solidity, a mean intensity entropy, and a median minor axis length. A standard deviation of the CoLIAGe information measure 1 possibly reflects the fact that malignant clusters have a larger variance in the magnitude of local textural correlations.

As shown in table 1400 of FIG. 14A, the best performance of a nuclear shape and texture classifier in differentiating malignant and benign clusters was provided the LDA classifier with features selected from mRMR, achieving an AUC of 0.87±0.01, a sensitivity of 0.79±0.005, and a specificity of 0.82±0.03 on 100 iterations of three-fold cross-validation. In comparison to the texture shape, combining the top features from each feature family resulted in an improvement in sensitivity of 2% from 75% to 77% and training AUC by 1% from 85% to 86%

FIG. 14B illustrates a UMAP plot 1402 that depicts a diagnostic ability of the top five nuclear shape features. The UMAP plot 1402 shows improvement in cell cluster area separation when combining all feature families.

FIG. 14C illustrates a graph 1404 showing exemplary Receiver operating characteristic curves for each classifier trained on a mix of five shape and texture features selected by mRMR. Graph 1404 demonstrates the validation AUC of each classifier with features selected from mRMR, with LDA having the highest AUC of 0.79.

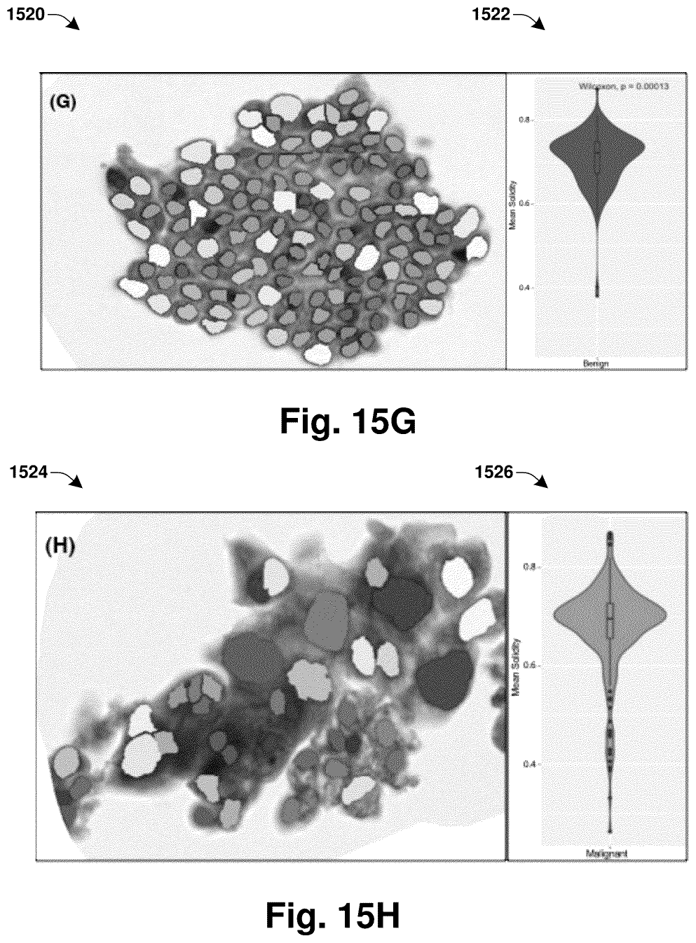

FIGS. 15A-15H illustrate graphical depictions of different diagnostic features used in some embodiments of a disclosed method and/or apparatus of generating a cytological diagnosis using a diagnostic combination of top five diagnostic nuclear shape and cluster texture features.

FIGS. 15A-15B illustrate cell cluster images for benign cell cluster areas 1500 and malignant cell cluster areas 1502. FIG. 15A illustrates some embodiments of a cell cluster image for benign cell cluster areas 1500 magnified at 40× magnification. FIG. 15B illustrates some embodiments of a cell cluster image for malignant cell cluster areas 1502 magnified at 40× magnification.

FIGS. 15C-15D illustrate feature heat maps and distribution visualizations for benign cell clusters and malignant cell cluster areas. FIG. 15C illustrates some embodiments of a feature heat map 1504 and a distribution visualization 1506 for a benign cell cluster area. FIG. 15D illustrates some embodiments of a feature heat map 1508 and a distribution visualization 1510 for a malignant cell cluster area.

The feature heat maps, 1504 and 1508, shown in FIGS. 15C-15D visually depict a “Mean CoLIAGe information measure 1” feature. The heat maps suggest that larger areas of highly correlated pixels reflect regions of textural homogeneity. These regions of larger energy sums on the heat maps represent textural homogeneity, likely reflecting regions of dense chromatin distribution and showing evidence of nuclear overcrowding. The malignant cluster area in FIG. 15D shows more regions of stronger correlation than the corresponding benign cluster areas in FIG. 15C, in turn, reflecting more homogeneous nuclear regions. This appears to suggest evidence of hyperchromasia and hypochromasia, one of the primary criteria of malignancy.

The yellow regions inside the nuclei encircled by darker red regions highlight the presence of nucleoli being more prominent. Thus, the distribution of dark spots contributes to poorer local pixel intensity correlation and a higher deviation in correlation for malignant clusters. Regions of higher correlation in the malignant cytoplasm may be reflecting abnormal secretion of proteins. Particularly around the edges of malignant nuclei, the presence of more dominant red edges packed around nuclear boundaries due to greater textural correlation in malignant clusters might reflect the presence of marginal chromatin in malignant phenotypes and the presence of three-dimensional nuclear overcrowding of malignant nuclei found on the BDB specimens as a result of erratic growth of nuclei on top of one another. In contrast to malignant nuclei, benign nuclei are arranged in sheets, in turn reflecting a more orderly arrangement of nuclei. The blue regions (area of a lower likelihood of similar gray level pixel probabilities) within the malignant nuclei are likely a consequence of the denser distribution of chromatin within the nuclei. The yellow regions in the benign cluster represent the cytoplasmic region, in turn reflecting a sparse distribution of cytoplasmic contents through the cytoplasm. Conversely, the cytoplasm in malignant clusters presents with a higher degree of correlation, which in turn suggests a more heterogeneous and disorderly distribution of cytoplasmic contents.

FIGS. 15E-15F illustrate Mean Haralick contrast entropies for benign cell clusters and malignant cell clusters. FIG. 15E illustrates some embodiments of a Mean Haralick contrast entropy 1512 and a distribution visualization 1514 for a benign cell cluster. FIG. 15F illustrates some embodiments of a Mean Haralick contrast entropy 1516 and a distribution visualization 1518 for a malignant cell cluster.

FIGS. 15G-15H illustrate mean solidity of nuclei, 1520 and 1524, and corresponding distribution visualizations, 1522 and 1526. The mean solidity is a nondimensional metric that measures an irregularity of polygons. Benign nuclei have a much higher mean solidity than that of malignant nuclei. This could indicate that the shape of benign nuclei is in general more circular/ovular than their malignant counterparts. In addition, the minor axis length of each nucleus of a benign cluster is in general lower compared with a malignant cell cluster area. Not only is the mean value of the minor axis lengths lower than the corresponding benign cell cluster areas, but the variability in these quantitative metrics is much smaller compared with the malignant cell cluster area. The larger minor axis length could reflect the fact that malignant nuclei are generally larger in volume and have a larger aspect ratio than their benign counterparts.

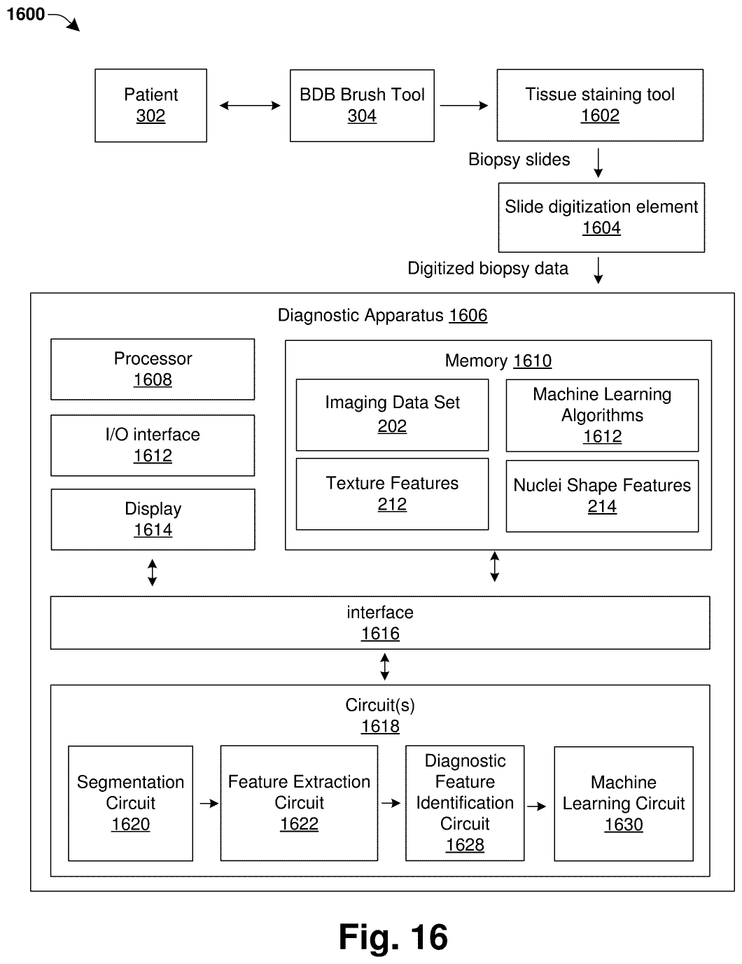

FIG. 16 illustrates a block diagram of some embodiments of an apparatus 1600 comprising a machine learning model configured to generate a cytological diagnosis using shape and texture features associated with epithelial cells obtained from the bile duct of a patient.

The apparatus 1600 comprises a diagnostic apparatus 1606. The diagnostic apparatus 1606 is coupled to a slide digitization element 1604 that is configured to obtain digitized pathology images (e.g., whole slide images) of epithelial cells collected from a patient 302 having a pancreaticobiliary disorder (e.g., including biliary adenocarcinomas and cholangiocarcinomas). In some embodiments, one or more tissue samples may be obtained by a BDB procedure and stained using a tissue staining tool 1602.

The diagnostic apparatus 1606 comprises a processor 1608 and a memory 1610. The processor 1608 can, in various embodiments, comprise circuitry such as, but not limited to, one or more single-core or multi-core processors. The processor 1608 can include any combination of general-purpose processors and dedicated processors (e.g., graphics processors, application processors, etc.). The processor(s) 1608 can be coupled with and/or can comprise memory (e.g., memory 1610) or storage and can be configured to execute instructions stored in the memory 1610 or storage to enable various apparatus, applications, or operating systems to perform operations and/or methods discussed herein.

Memory 1610 can be configured to store an imaging data set 202 comprising digitized pathology images for a plurality of patients having and/or suspected to have a pancreaticobiliary disorder. The digitized images may comprise a plurality of pixels, each pixel having an associated intensity. In some additional embodiments, the digitized pathology images may be stored in the memory 1610 as one or more training sets and/or one or more testing sets for training a machine learning classifier and/or one or more validation sets (e.g., test sets).

The diagnostic apparatus 1606 also comprises an input/output (I/O) interface 1612 (e.g., associated with one or more I/O devices), a display 1614, one or more circuits 1618, and an interface 1616 that connects the processor 1608, the memory 1610, the I/O interface 1612, the display 1614, and the one or more circuits 1618. The I/O interface 1612 can be configured to transfer data between the memory 1610, the processor 1608, the one or more circuits 1618, and external devices (e.g., slide digitization element 1604).

In some embodiments, the one or more circuits 1618 may comprise hardware components. In other embodiments, the one or more circuits 1618 may comprise software components. The one or more circuits 1618 can comprise a segmentation circuit 1620 configured to perform a segmentation operation on one or more digitized pathology images within the imaging data set 202 to identify segmented nuclei and non-nuclei regions. In some additional embodiments, the one or more circuits 1618 may further comprise a feature extraction circuit 1622 configured to extract a plurality of texture features 212 from the segmented nuclei and the non-nuclei regions and to extract a plurality of nuclear shape features 214 of the segmented nuclei. In some additional embodiments, the one or more circuits 1618 may further comprise a diagnostic feature identification circuit 1628 configured to identify diagnostic features from the plurality of texture features 212 and the plurality of nuclear shape features 214. In some additional embodiments, the one or more circuits 1618 may further comprise a machine learning circuit 1630 configured to classify the epithelial cells as malignant or non-malignant based upon the diagnostic features comprising one or more of the plurality of texture features 212 and one or more of the plurality of nuclear shape features 214.

Therefore, the present disclosure relates to a method and/or apparatus configured to generate a cytological diagnosis (e.g., to diagnose a presence of pancreatic and/or biliary tract adenocarcinoma) using nuclear shape and texture features extracted from digitized pathology images of epithelial cell clusters collected using a bile duct brushing procedure.

In some embodiments, the present disclosure relates to a method. The method includes accessing one or more digitized pathology images of a cell cluster area having epithelial cells obtained from a bile duct of a patient having a bile duct stricture; segmenting the cell cluster area to identify segmented nuclei and non-nuclei regions; extracting a plurality of texture features from the segmented nuclei and the non-nuclei regions; extracting a plurality of nuclear shape features from the segmented nuclei; and providing one or more of the plurality of nuclear shape features and the plurality of texture features to a machine learning model configured to generate a cytological diagnosis of the epithelial cells within the cell cluster area.

In other embodiments, the present disclosure relates to a non-transitory computer-readable medium storing computer-executable instructions that, when executed, cause a processor to perform operations. The operations include accessing one or more digitized pathology images, the one or more digitized pathology images having a cell cluster area including epithelial cells obtained from a stricture within a bile duct of a patient; segmenting the cell cluster area to identify segmented nuclei and non-nuclei regions; extracting a plurality of texture features from the segmented nuclei and the non-nuclei regions; extracting a plurality of nuclear shape features from the segmented nuclei; identifying a plurality of diagnostic features from the plurality of texture features and the plurality of nuclear shape features; and providing the plurality of diagnostic features to a machine learning model configured to classify the cell cluster area as malignant or non-malignant

In other embodiments, the present disclosure relates to an apparatus. The apparatus includes a memory configured to store one or more digitized pathology images, the one or more digitized pathology images having a cell cluster area including epithelial cells obtained from a stricture within a bile duct of a patient; a plurality of circuits, including: a segmentation circuit configured to segment the one or more digitized pathology images to identify segmented nuclei and non-nuclei regions; a feature extraction circuit configured to extract a plurality of texture features from the segmented nuclei and the non-nuclei regions and to extract a plurality of nuclear shape features from the segmented nuclei; and a machine learning circuit configured to classify the epithelial cells as malignant or non-malignant based upon one or more of the plurality of texture features and one or more of the plurality of nuclear shape features.

Examples herein can include subject matter such as an apparatus, including a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system according to embodiments and examples described. References to “one embodiment”, “an embodiment”, “one example”, and “an example” indicate that the embodiment(s) or example(s) so described may include a particular feature, structure, characteristic, property, element, or limitation, but that not every embodiment or example necessarily includes that feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing one or more digitized pathology images of a cell cluster area comprising epithelial cells obtained from a bile duct of a patient having a bile duct stricture;
segmenting the cell cluster area to identify segmented nuclei and non-nuclei regions;
extracting a plurality of texture features from the segmented nuclei and the non-nuclei regions;
extracting a plurality of nuclear shape features from the segmented nuclei; and
providing one or more of the plurality of nuclear shape features and the plurality of texture features to a machine learning model configured to generate a cytological diagnosis of the epithelial cells within the cell cluster area.

2. The method of claim 1, further comprising:
inserting a cytology brush into the bile duct of the patient to obtain a brush specimen comprising the epithelial cells from the bile duct stricture;
placing the epithelial cells on a slide; and
obtaining an image of the slide to form a whole slide image, wherein the one or more digitized pathology images comprise the whole slide image.

3. The method of claim 2, further comprising:
removing effects of illumination of the one or more digitized pathology images by moving a fixed size window iteratively for respective ones of a plurality of pixels and subtracting a local average associated with the fixed size window from the one or more digitized pathology images.

4. The method of claim 1, further comprising:
providing one or more of the plurality of nuclear shape features and one or more of the plurality of texture features to the machine learning model.

5. The method of claim 1, wherein the plurality of texture features include one or more of Gabor features, Law's features, Haralick features, and CoLIAGe (Co-occurrence of Local Anisotropic Gradient Orientations) features.

6. The method of claim 1,
wherein the plurality of texture features are computed for each pixel within the one or more digitized pathology images; and
wherein statistical measures are computed for each texture feature over the cell cluster area.

7. The method of claim 6, wherein the machine learning model is able to classify atypical epithelial cells within the cell cluster area as being benign or malignant with a specificity of greater than 95%.

8. The method of claim 1, wherein the plurality of texture features are determined at an aggregate level on the cell cluster area to investigate interplay between the segmented nuclei and cytoplasm surrounding the segmented nuclei.

9. The method of claim 1, further comprising:
forming an elliptical bounding box around each of the segmented nuclei, wherein the elliptical bounding box is used to compute one or more of the plurality of nuclear shape features.

10. The method of claim 9, wherein the plurality of nuclear shape features comprise one or more of an area, a major axis length of the elliptical bounding box, a minor axis length of the elliptical bounding box, an orientation of a major axis length, an equivalent diameter, a solidity, and a perimeter.

11. The method of claim 1, wherein the plurality of nuclear shape features comprise a nuclei-to-cytoplasm ratio determined for the cell cluster area by dividing a total nuclear area of the cell cluster area by a non-nuclear area of the cell cluster area.

12. The method of claim 1, further comprising:
identifying a plurality of diagnostic features from the plurality of texture features and the plurality of nuclear shape features.

13. The method of claim 12, wherein the machine learning model has a higher specificity in identify atypical epithelial cells within the cell cluster area than in identifying non-atypical epithelial cells within the cell cluster area.

14. The method of claim 13, wherein the plurality of diagnostic features comprise a mean contrast entropy, a standard deviation of a CoLIAGe (Co-occurrence of Local Anisotropic Gradient Orientations) correlation information measure 1, a mean solidity, a mean intensity entropy, and a median minor axis length.

15. A non-transitory computer-readable medium storing computer-executable instructions that, when executed, cause a processor to perform operations, comprising:
accessing one or more digitized pathology images, the one or more digitized pathology images comprising a cell cluster area including epithelial cells obtained from a stricture within a bile duct of a patient;
segmenting the cell cluster area to identify segmented nuclei and non-nuclei regions;
extracting a plurality of texture features from the segmented nuclei and the non-nuclei regions;
extracting a plurality of nuclear shape features from the segmented nuclei;
identifying a plurality of diagnostic features from the plurality of texture features and the plurality of nuclear shape features; and
providing the plurality of diagnostic features to a machine learning model configured to classify the cell cluster area as malignant or non-malignant.

16. The non-transitory computer-readable medium of claim 15, wherein the plurality of texture features including one or more of Gabor features, Law's features, Haralick features, and CoLIAGe (Co-occurrence of Local Anisotropic Gradient Orientations) features.

17. The non-transitory computer-readable medium of claim 15, further comprising:
forming an elliptical bounding box around each of the segmented nuclei, wherein the elliptical bounding box is used to compute one or more of the plurality of nuclear shape features including an eccentricity of the segmented nuclei.

18. The non-transitory computer-readable medium of claim 15, further comprising:
forming an elliptical bounding box around each of the segmented nuclei; and
wherein the plurality of nuclear shape features comprise one or more of an area, a major axis length of the elliptical bounding box, a minor axis length of the elliptical bounding box, an orientation of a major axis length with respect to a horizontal, an equivalent diameter, a solidity, and a perimeter.

19. An apparatus, comprising:
a memory configured to store one or more digitized pathology images, the one or more digitized pathology images comprising a cell cluster area including epithelial cells obtained from a stricture within a bile duct of a patient;
a plurality of circuits, comprising:
a segmentation circuit configured to segment the one or more digitized pathology images to identify segmented nuclei and non-nuclei regions;
a feature extraction circuit configured to extract a plurality of texture features from the segmented nuclei and the non-nuclei regions and to extract a plurality of nuclear shape features from the segmented nuclei; and
a machine learning circuit configured to classify the epithelial cells as malignant or non-malignant based upon one or more of the plurality of texture features and one or more of the plurality of nuclear shape features.

20. The apparatus of claim 19, further comprising:
a diagnostic feature identification circuit configured to identify a plurality of diagnostic features from the plurality of texture features and the plurality of nuclear shape features, wherein the machine learning circuit is configured to operate upon the plurality of diagnostic features to classify the cell cluster area.
